(19) United States
(12) Patent Application Publication
(10) Pub. No.: US 2020/0321084 A1
(43) Pub. Date: Oct. 8, 2020

(54) DEVICE, SYSTEM, AND METHOD FOR OPTIMIZING PATHOLOGY WORKFLOWS

(71) Applicant: KONINKLIJKE PHILIPS N.V., EINDHOVEN (NL)

(72) Inventors: Anca Ioana Daniela BUCUR, WAALRE (NL); Jasper Johannes Adrianus VAN LEEUWEN, EINDHOVEN (NL)

(21) Appl. No.: 15/733,118

(22) PCT Filed: Nov. 12, 2018
(86) PCT No.: PCT/EP2018/080850

§ 371 Date: May 20, 2020

Related U.S. Application Data
(60) Provisional application No. 62/589,713, filed on Nov. 22, 2017.

(51) Int. Cl. G16H 10/40; G16H 50/70; G16H 40/20; G16H 10/60

(52) U.S. Cl. CPC G16H 10/40; G16H 10/60; G16H 40/20; G16H 50/70

(57) ABSTRACT

A device, system, and method optimizes pathology workflows. The method performed at a workflow server includes receiving a plurality of digital slides associated with a pathology case, the pathology case associated with first information characterizing the pathology case. The method includes generating second information based on an analysis of the digital slides, the second information characterizing the digital slides. The method includes determining a plurality of tasks used in completing the pathology case based on the first and second information. The method includes determining a task performer to be assigned to perform a select one of the tasks. The method includes dispatching an assignment to the task performer corresponding to the selected task.

### BACKGROUND INFORMATION

Pathology generally relates to the study of diseases where a pathologist may diagnose and/or track a disease by analyzing samples of a patient including tissues, cells, body fluid, or a combination thereof. In a conventional approach to performing the pathologist duties, an analogue pathology procedure may be used where pathologists view pathology slides through a microscope or other viewing tool. For example, a physical slide of an actual sample is prepared for the pathologist to analyze. However, the use of the physical pathology slides requires that either the slides travel to the pathologist or the pathologist travels to the slides to complete the diagnosis.

To address the co-location requirement, another approach to performing the pathologist duties is a digital pathology procedure. The digital pathology procedure may be used where pathology slides of samples of a patient are digitized (e.g., by a scanner) to allow the pathologist to subsequently assess and diagnose the sample by viewing the digital copy of the pathology slide. The advent of the digital pathology procedure introduces the capability of decoupling the pathologist from the location of the physical slides.

Using conventional approaches in both analogue and digital pathology procedures, a part of the workflow of a pathology case is steered by decisions of the pathologist. For example, the pathologist is required to make manual decisions along certain aspects of the workflow. In a first example, after an initial assessment of the pathology case (e.g., by viewing a preliminary set of physical or digital pathology slides), the pathologist may determine that an expert pathologist or specialist is required to further diagnose the pathology case. In a second example, the pathologist may determine that the pathology case is relevant for a further purpose such as a teaching purpose. In a third example, the pathologist may determine that additional tests and/or stains may be required to continue diagnosing the pathology case. In view of these manual decision possibilities, the workflow may require more time and become less efficient to complete the pathology workflow.

The above noted issues assume that the criteria of having to initially assign or dispatch the pathology case to a pathologist has been completed. However, this process of assigning the pathologist may also include issues that disrupt the efficiency of a workflow in completing the pathology case. Specifically, using conventional approaches in both analogue and digital pathology procedures, a part of the workflow of a pathology case includes an administrator or other assignment entity/user to determine which pathologist to select for a pathology case or portion thereof. However, there may be a large number of variables involved in determining how a pathology case is assigned to a pathologist (e.g., organ type, extraction method, clinical question, place in the workflow of the pathology case, availability of the pathologist, expertise of the pathologist, role of the pathologist, etc.). Due to this large number of variables and how to consider these variables, especially in combination, those skilled in the art will understand that the assignment of pathology cases to pathologists may not be performed in an optimal or efficient manner.

In a further aspect with regard to determining a selection of a pathologist to be assigned a pathology case, the determination may also be made based on a set of defined policies that may be specific to a lab or pathology entity that captures the context (e.g., the samples). For example, the policies may be associated with characteristics of the pathologists (e.g., expertise, role, available time, etc.) and characteristics of the pathology cases (e.g., type, complexity, expected diagnosis time, etc.) as well as goals of the lab (e.g., fairness, throughput, turnaround, cost/income, resource utilization, timeliness, etc.). In the analogue pathology procedure, these policies may be defined in documents and implemented manually (e.g., by a technician of the lab). However, there is no computerized support or automated functionality integrating the relevant workflows related to the diagnosis tasks (e.g., quality control, consultation, resident versus attending pathologist contribution to the diagnosis, etc.) to support efficient task distribution that implements the relevant policies in each workflow. Even if the digital pathology procedure were to incorporate further operations that provide an automated mechanism for task distribution and corresponding workflows to be computerized and the policies to be implemented in software, the attributes of the entities to be used in defined domain models corresponding to the policies would have to be used by optimization algorithms that apply the policies (e.g., defined as rules and constraints on the model) to propose solutions for task distribution that improve on the defined goals. However, as described below, even assuming the automated operation were available, such a functionality requires incorporation of a plurality of different conditions and criteria.

When optimizing a pathology workflow, one or more optimization goals may be selected for consideration in completing a pathology case. For example, in a diagnosis workflow, a fairness of the allocation, the throughput of pathology cases, a turnaround of the pathology cases, a resource utilization of a pathology entity, etc. may be defined as goals. However, in the course of defining the goals and selection thereof, a combination of the goals may partially conflict with one another. When this occurs, trade-offs of at least one goal may have to be defined. To balance a workload of the pathologists working in a lab and to improve an overall performance of the lab, it may be necessary to associate to each of several case attributes (e.g., average diagnosis time or complexity) that reliably express an effort, a time, a cost, etc. for diagnosis. These attributes may be used to propose solutions that improve on the selected goals.

However, implementing the policies and implicitly the corresponding optimization goals for the task distribution within the various workflows of a lab may require a significant amount of time and yield a large number of complex and sometimes overlapping or conflicting rules and goals. Furthermore, this operation scales poorly when aiming to deploy an implementation at a large number of labs with respective policies and goals. Even if a solution were devised where an implementation of entire lab policies serves as templates to be selected and adapted to each new lab, the overall operation may be substantially difficult to detect conflicts among rules, set the correct trade-offs among scores that would enable reaching the best optimization solution, validate and debug the adapted solution for new labs, etc.

In another element in assigning or dispatching pathology cases to pathologists, the effort required to diagnose a pathology case may vary widely and depend on the case itself (e.g., the clinical question, the type of organ, number and type of pathology slides that compose the case, positive or negative diagnosis result, etc.) and/or on the particular pathologist (e.g., parameterized by expertise, experience, skill, etc.). As noted above, when optimizing the diagnosis workflow of a pathology case, at least one optimization goal may be selected (e.g., the fairness of the allocation, the throughput of cases, the turnaround of cases, the resource allocation for the cases, the timeless in completing the cases, etc.). Thus, in addition to optimizing the efficiency of completing a pathology case as an optimization goal, there may be another optimization goal to optimize the efficiency of a pathology entity and the pathologists.

In balancing a workload of the pathologists associated with a pathology entity and to improve an individual and overall performance, each case must be associated with attributes (e.g., average diagnosis time, complexity, etc.) that reliably express factors involved in diagnosis (e.g., effort, time, cost, etc.). In a conventional approach, the pathology entity may propose averages for diagnosis type per case type that are applied to all pathologists. However, in practice, the actual diagnosis time may vary greatly depending on the expertise of each pathologist and on the complexity of the specific case (not only the complexity of the class). The overall specialty measure may only be a rough estimation with differences among pathologists that also evolve over time (e.g., pathologists gain new expertise and recall of previous cases also contribute to optimization). Although the digital pathology procedure may enable new information to be collected (e.g., both scanner and image management systems) to optimize a diagnostic workflow (e.g., to bring results back to patients faster), current approaches that use averages to estimate diagnosis time, while aligned with the estimates used for reimbursement, lead to inaccurate predictors of throughput and turnaround.

### SUMMARY

The exemplary embodiments are directed to a method, comprising: at a workflow sever: receiving a plurality of digital slides associated with a pathology case, the pathology case associated with first information characterizing the pathology case; generating second information based on an analysis of the digital slides, the second information characterizing the digital slides; determining a plurality of tasks used in completing the pathology case based on the first and second information; determining a task performer to be assigned to perform a select one of the tasks; and dispatching an assignment to the task performer corresponding to the selected task.

The exemplary embodiments are directed to a workflow server, comprising: a transceiver communicating via a communications network, the transceiver configured to receive a plurality of digital slides associated with a pathology case, the pathology case associated with first information characterizing the pathology case; a memory storing an executable program; and a processor that executes the executable program that causes the processor to perform operations, comprising: generating second information based on an analysis of the digital slides, the second information characterizing the digital slides; determining a plurality of tasks used in completing the pathology case based on the first and second information; determining a task performer to be assigned to perform a select one of the tasks; and dispatching an assignment to the task performer corresponding to the selected task.

The exemplary embodiments are directed to a method, comprising: at a workflow server: receiving a plurality of digital slides associated with a plurality of pathology cases, the pathology cases associated with respective first information characterizing the corresponding pathology case; generating second information based on an analysis of the digital slides, the second information characterizing the digital slides; determining a plurality of tasks to be completed in a window of time, the plurality of tasks associated with the pathology cases based on the first and second information; determining a task performer to be assigned to perform a select one of the tasks; and dispatching an assignment to the task performer corresponding to the selected task, wherein the assignment is associated with an optimization goal of one of fairness, throughput, turnaround, resource allocation, timeliness, or a combination thereof.

### BRIEF DESCRIPTION OF THE DRAWINGS

FIG. 1 shows a system according to the exemplary embodiments.

FIG. 2 shows a workflow server of FIG. 1 according to the exemplary embodiments.

FIG. 3 shows an overall method for automatically completing a pathology case according to the exemplary embodiments.

FIG. 4 shows a method for generating policies for assigning a pathologist to a pathology case according to the exemplary embodiments.

FIG. 5 shows a method for assigning a pathologist to a pathology case according to the exemplary embodiments.

### DETAILED DESCRIPTION

The exemplary embodiments may be further understood with reference to the following description and the related appended drawings, wherein like elements are provided with the same reference numerals. The exemplary embodiments are related to a device, a system, and a method for optimizing a workflow for completing a pathology case. The exemplary embodiments are configured to automatically determine a plurality of selections along the workflow pathway. Specifically, the exemplary embodiments provide an overall process that determines the manner in which a pathology case is to be completed. Along the overall process, a workflow may be divided into tasks to which a pathologist is assigned. The exemplary embodiments provide a mechanism by which policies are generated to define a manner in which optimization goals associated with dispatching task assignments are met. The exemplary embodiments further provide a mechanism by which the task assignments are determined based on the policies and dynamically determined, current characteristics of the available pathologists. As will be described in further detail below, the exemplary embodiments utilize a digital pathology procedure and provide features for the selections along the workflow pathway to be determined in an automated manner.

It is noted that the exemplary embodiments are described with respect to optimizing the workflow for completing a pathology case. However, the use of the pathology case and workflow associated therewith are only exemplary. The exemplary embodiments may be modified to be used with any medical case (e.g., an image acquisition procedure) or non-medically related case in which a workflow is used to complete the case, particularly through determining tasks associated with the workflow and assigning a user to the task to meet optimization goals. The exemplary embodiments are also described with respect to generating policies associated with assigning a pathologist to a task. However, the implementation to generate policies for assigning a pathologist to a task is only exemplary. The exemplary embodiments may be modified to be used in generating policies for any entity such that conflicts among rules or models are resolved to achieve the policies. The exemplary embodiments are further described with respect to determining how pathologists are assigned to different tasks in the workflow. However, the assignment of pathologists to tasks is only exemplary. The exemplary embodiments may be modified to be used in determining a match to achieve an optimization goal. The exemplary embodiments may also determine how any task performer is assigned the different tasks in the workflow and is not limited to only pathologists (e.g., lab technicians, archivists, etc.). However, for illustrative purposes, the exemplary embodiments are described herein with particular regard to assigning pathologists.

The exemplary embodiments provide a mechanism that generates and uses information that was not available when completing a pathology case under an analogue pathology procedure. Specifically, the exemplary embodiments are directed to a digital pathology procedure such that additional information generated by leveraging an availability of digital slides of samples are utilized. As will be described in detail below, algorithms may be applied on the digital slides to drive workflow selection, workflow execution and resource assignment (e.g., a pathologist, an equipment, etc.). The exemplary embodiments provide an automated analysis of the digital slides which may be used to drive or optimize workflow decisions. For example, three different areas may be distinguished: (1) selection of a workflow, (2) selection of paths at decision points in a workflow, and (3) assessment of resource requirements (e.g., required diagnosis time by a pathologist).

The exemplary embodiments take advantage of a location decoupling associated with the analogue pathology procedure. The location decoupling introduces the opportunity to change and optimize the workflow of pathology cases after the digital slides have been created and scanned in the laboratory. For example, the workflow of pathology cases may allow for more insights to be generated and improve efficiency as well as quality. Furthermore, the analysis of digital pathology slides may be used to steer the workflow and improve resource allocation. The decoupling of the location requirement may also have an impact on a planning and scheduling of cases such as which pathologist is to diagnose a sample. In a digital pathology laboratory, a pathologist associated with a pathology entity may be requested to assess cases arriving from a variety of sources in an internal manner from within the pathology entity (e.g., a routine diagnosis) as well as an external manner from outside the pathology entity (e.g., second opinion requests). Those skilled in the art will appreciate that the location decoupling may enable more flexibility in completing a pathology case. However, conventional approaches may only utilize the location decoupling aspect of digital pathology procedures while still maintaining all remaining manners of completing the pathology case as an analogue pathology procedure.

The exemplary embodiments also provide a change in the granularity of handling a pathology case. Conventional approaches rely on assigning an entire pathology case to a single pathologist. Accordingly, the selected pathologist performs all the necessary operations to complete the pathology case or determines and delegates other operations (e.g., when a particular expertise is required). However, the exemplary embodiments introduce a manner of determining one or more tasks associated with a pathology case (e.g., based on the information derived from the digital slides). Accordingly, each task may have its own considerations and criteria that may be used to determine which pathologist is to be assigned the task.

As will be described in detail below, the exemplary embodiments may provide an automated resource planner that manages and/or assigns the resources (e.g., a pathologist) used to perform a task in a pathology workflow. The resource planner may use information from the automated analysis of the pathology slides to better assess the resource requirements. For example, an IHC HER2 scoring algorithm may indicate a likely equivocal score such that a workflow may be triggered involving an ordering of a HER2 FISH test. Subsequently, the resource planner may use this information to better assess the expected diagnosis time required for diagnosing the case (as the ordered HER2 FISH test has impact on the diagnosis activity).

The automated resource planner may also incorporate models at an atomic level in determining how to assign resources. The exemplary embodiments provide a solution that efficiently scales up a modelling, an implementation, and a subsequent adaptation of policies as well as increases a reuse of rules and goals and helps avoid errors when defining and managing a large number of rules and goals. The exemplary embodiments may also use an implementation of entire policies of a pathology entity as templates to be selected and adapted for another pathology entity. In this alternative approach, the exemplary embodiments may be configured to detect conflicts among rules, set correct trade-offs among scores that enable reaching an optimization solution, and validate/debug an adapted solution for further pathology entities. The exemplary embodiments additionally improve an accuracy of a domain model and constraint values underlying an automatic assignation of pathology cases or tasks, thereby enabling more accurate performance analysis and goal optimization leading to higher and more reliable performance gains.

The exemplary embodiments may further provide a mechanism to associate a pathology case with a workflow and corresponding tasks. Conventional approaches utilize a singular view of a pathology case in which a single case status is used (e.g., in preparation, ready for review, action required, finished, etc.). In contrast, the exemplary embodiments may depart from this characterization of a pathology case to an association with the workflow and the tasks. As will be described in further detail below, this association of the pathology case may provide improved manners of achieving optimization goals relative to the singular characterization used by conventional approaches.

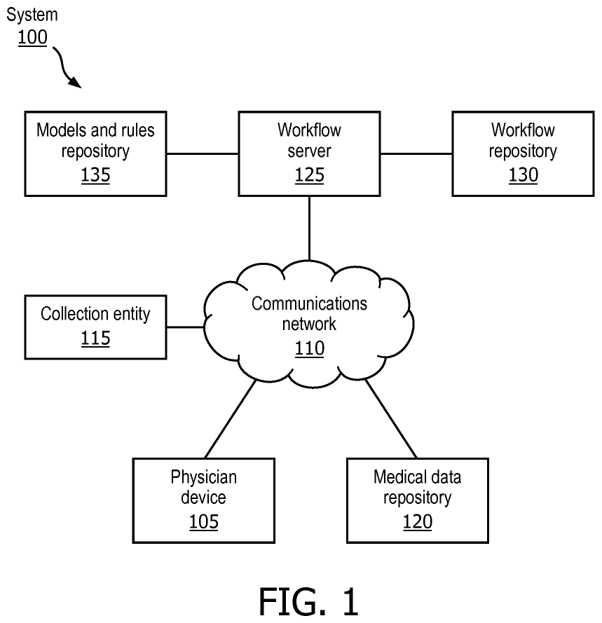

FIG. 1 shows a system 100 according to the exemplary embodiments. The system 100 relates to a communication between various components involved in completing a pathology case. Specifically, the system 100 may relate to a scenario when a patient provides a sample to be diagnosed, the sample is used to create digital slides, the digital slides are used in the diagnosis, and the system 100 determines the manner in which the pathology case should be completed based at least in part on the digital slides. The system 100 may include a physician device 105, a communications network 110, and a collection entity 115. The system 100 may also have access to various sources of information that may be used in completing a pathology case. The various sources of information including any information received from the physician device 105 and/or the collection entity 115 may be represented by the medical data repository 120. The system 100 may further include a workflow server 125 that determines the manner in which the pathology case is completed, specifically via assignment of tasks associated with a workflow corresponding to a selected pathology case. In performing its functionality, the workflow server 125 may utilize data included in a workflow repository 130 and a model and rules repository 135.

The physician device 105 may represent any electronic device that is configured to perform the functionalities associated with a physician. Specifically, the physician device 105 may be utilized by a pathologist. For example, the physician device 105 may be a portable device such as a tablet, a laptop, etc. or a stationary device such as a desktop terminal. The physician device 105 may include the necessary hardware, software, and/or firmware to perform the various operations associated with medical treatment, particularly in tracking a pathology case based on data exchange with the workflow server 125. The physician device 105 may also include the required connectivity hardware, software, and firmware (e.g., transceiver) to establish a connection with the communications network 110 to further establish a connection with the other components of the system 100. The physician device 105 may further be configured to receive digital slides of samples and configured to show the digital slides to the pathologist in diagnosing the sample.

The physician device 105 may be configured to enable the pathologist to perform the various operations associated with medical treatment or diagnosis. For example, the physician device 105 may receive a schedule of upcoming diagnoses to be performed from the workflow server 125. In this manner, the pathologist may receive the corresponding digital slides to be diagnosed. In another example, the physician device 105 may be used to provide results of a diagnosis or other information associated with the diagnosis to the workflow server 125.

It is noted that the physician device 105 may also represent an administrator device or other user device who may provide inputs to the workflow server 125 to define the manners in which pathology cases are completed. As will be described in further detail below, the workflow server 125 may utilize various information including manually entered inputs in defining the manners of case completion. For example, the administrator or user of the workflow server 125 may also be a pathologist. In another example, the user may be a planner or engineer with the knowledge of how to incorporate the various features of the exemplary embodiments.

The communications network 110 may be configured to communicatively connect the various components of the system 100 to exchange data. The communications network 110 may represent any single or plurality of networks used by the components of the system 100 to communicate with one another. For example, if the physician device 105 is used at a hospital, the communications network 110 may include a private network with which the physician device 105 may initially connect (e.g. a hospital network). The private network may connect to a network of an Internet Service Provider to connect to the Internet. Subsequently, through the Internet, a connection may be established to other electronic devices. For example, the workflow server 125 may be remote relative to the hospital but may be connected to the Internet. Thus, the physician device 105 may be communicatively connected to the workflow server 125. It should be noted that the communications network 110 and all networks that may be included therein may be any type of network. For example, the communications network 110 may be a local area network (LAN), a wide area network (WAN), a virtual LAN (VLAN), a WiFi network, a HotSpot, a cellular network (e.g., 3G, 4G, Long Term Evolution (LTE), etc.), a cloud network, a wired form of these networks, a wireless form of these networks, a combined wired/wireless form of these networks, etc.

The collection entity 115 may represent any person or organization that collects samples and generates the digital slides of the samples. For example, the collection entity 115 may receive samples from a physician's office, a lab, etc. The collection entity 115 may digitize the sample into an appropriate slide. For example, a tissue sample may be oriented or shaped into a cross-sectional view that is lighted and/or stained. An image of the view may be captured and formatted into a digital slide. Those skilled in the art will understand that the collection entity 115 may use any mechanism in which to convert a physical sample into a digital slide. The collection entity 115 may include the required connectivity hardware, software, and firmware (e.g., transceiver) to establish a connection with the communications network 110 to further establish a connection with the other components of the system 100. For example, the digital slides that are created by the collection entity 115 may be stored in the medical data repository 120 or transmitted to the workflow server 125.

The medical data repository 120 may be a repository of medical data that may be queried for information pertaining to patients. In a first example, the medical data repository 120 may be directed to patient histories where each patient may have an electronic medical record (EMR) used to track the different procedures, treatments, visits, etc. of the patient and also track diagnoses associated with different tasks of a pathology case. In a second example, the medical data repository 120 may be directed to storing samples, images, or digital slides as well as associated information. In a particular example, when related to pathology, this aspect of the medical data repository 120 may be substantially similar to a Lab Information System. When related to image acquisition, this aspect of the medical data repository 120 may be substantially similar to a Radiology Information System. In a third example, the medical data repository 120 may be directed to tracking and logging protocols and/or steps in performing particular procedures such as with pathology cases. In a particular example, when related to pathology, this aspect of the medical data repository 120 may be substantially similar to an Image Management System. When related to image acquisition, this aspect of the medical data repository 120 may be substantially similar to a Picture Archiving and Communication System.

The workflow server 125 may be a component of the system 100 that performs functionalities associated with determining how to complete a pathology case according to the exemplary embodiments. As will be described in further detail below, the workflow server 125 may include a mechanism in which an overall procedure is performed to complete the pathology case using an associated workflow and determined tasks within the workflow. Within the process of completing the pathology case, the workflow server 125 may include further mechanisms associated with defining optimization goals and policies to be used in completing the pathology case as well as selecting a pathologist to be assigned to a particular task of the workflow.

In performing its functionality, the workflow server 125 may utilize the workflow repository 130 and the model and rules repository 135. The workflow repository 130 may store a plurality of workflows that may be used in completing a pathology case. The workflows may be associated with various characteristics (e.g., keywords) such that a pathology case having matching characteristics may indicate the use of a selected workflow. The workflow repository 130 may also store a plurality of tasks associated with each workflow. The workflows and tasks stored in the workflow repository 130 may be determined based on, for example, historical pathology cases as stored in the medical data repository 120. The model and rules repository 135 may store a plurality of models (and/or rules) including atomic policy models and composite policy models that are selected to meet identified optimization goals. The models (and/or rules) stored in the model and rules repository 135 may be generated and/or determined based on, for example, historical performances of pathology cases and pathologists that are updated to be as relevant for a current pathology case being processed.

It is noted that the system 100 may include a plurality of physician devices 105, a plurality of collection entities 115, and a plurality of workflow servers 125. That is, many different physicians and collection entities may utilize or be associated with the system 100. There may also be many different workflow servers 125 that service different physician devices 105 and collection entities 115. For example, the workflow server 125 may be linked to a geographical region or a particular medical field. It is also noted that the storage capability and any associated functionalities of the medical data repository 120 being implemented in a single component of the system 100 is only exemplary. According to another exemplary embodiment, each functionality and corresponding storage capability of the medical data repository 120 may be incorporated into individual system components.

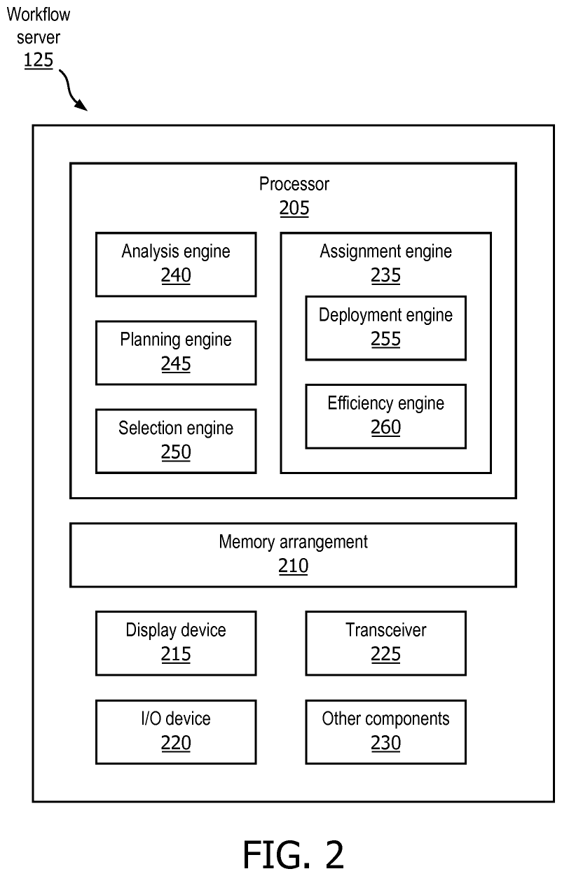

As described above, the workflow server 125 may determine the manner in which a pathology case is to be completed by determining an associated workflow, identifying tasks within the workflow, and assigning a pathologist to each of the tasks for completion. FIG. 2 shows the workflow server 125 of FIG. 1 according to the exemplary embodiments. The workflow server 125 may provide various functionalities in completing the pathology case. Although the workflow server 125 is described as a network component (specifically a server), the workflow server 125 may be embodied in a variety of hardware components such as a portable device (e.g., a tablet, a smartphone, a laptop, etc.), a stationary device (e.g., a desktop terminal), incorporated into the physician device 105 and/or the collection entity 115, incorporated into a website service, incorporated as a cloud device, etc. The workflow server 125 may include a processor 205, a memory arrangement 210, a display device 215, an input and output (I/O) device 220, a transceiver 225, and other components 230 (e.g., an imager, an audio I/O device, a battery, a data acquisition device, ports to electrically connect the workflow server 125 to other electronic devices, etc.).

The processor 205 may be configured to execute a plurality of applications of the workflow server 125. The processor 205 may utilize a plurality of engines including an assignment engine 235, an analysis engine 240, a planning engine 245, and a selection engine 250. The assignment engine 235 may also include a deployment engine 255 and an efficiency engine 260. The assignment engine 235 may receive various inputs to select a pathologist to be assigned a task. In performing this functionality, the deployment engine 255 may determine policies and optimization goals that are to be considered in assigning the pathologist to the task. The efficiency engine 260 may utilize the policies and optimization goals to identify a pathologist to be assigned to the task. The analysis engine 240 may receive and analyze the digital slides to generate additional information such as predicted case characteristics. The planning engine 245 may generate task allocations based on task allocation requests by following assignment rules (e.g., optimization goals, resource availability, etc.). The selection engine 250 may determine a workflow to be associated with a pathology case as well as determine tasks that are associated with the workflow.

It should be noted that the above applications and engines each being an application (e.g., a program) executed by the processor 205 is only exemplary. The functionality associated with the applications may also be represented as components of one or more multifunctional programs, a separate incorporated component of the workflow server 125 or may be a modular component coupled to the workflow server 125, e.g., an integrated circuit with or without firmware.

The memory 210 may be a hardware component configured to store data related to operations performed by the workflow server 125. Specifically, the memory 210 may store data related to the engines 235-260. The display device 215 may be a hardware component configured to show data to a user while the I/O device 220 may be a hardware component that enables the user to enter inputs. For example, an administrator of the workflow server 125 may maintain and update the functionalities of the workflow server 125 through user interfaces shown on the display device 215 with inputs entered with the I/O device 220. It should be noted that the display device 215 and the I/O device 220 may be separate components or integrated together such as a touchscreen. The transceiver 225 may be a hardware component configured to transmit and/or receive data via the communications network 110.

According to the exemplary embodiments, the workflow server 125 may perform various different operations to determine the manner in which a pathology case is to be completed. Specifically, the workflow server 125 via the engines 235-260 may utilize formal and executable models of desired pathology case workflows where the models are associated with a corresponding domain. By capturing all related information (e.g., task characteristics and pathologist information) necessary to assign tasks to pathologists and used by a resource planning component, an image analysis component, and constraints/policies/optimization goals with respect to task assignment, the exemplary embodiments may be enabled to determine how tasks are distributed by assigning a pathologist to support models of collaboration of desired complexity.

Initially, the workflow server 125 may receive an indication about a pathology case or at least one digital slide associated with the pathology case. This may be a trigger for the workflow server 125 to perform its functionality of determining the manner in which to complete the pathology case. For example, the workflow server 125 may monitor the medical data repository 120 that stores the digital slides from the collection entity 115. When new digital slides are identified, the workflow server 125 may request the digital slides. The digital slides may be associated with a specific pathology case (e.g., a marker or identification of the pathology case may be associated with the digital slide). In another manner, the workflow server 125 may analyze the digital slides and identify the associated pathology case by referencing other information in the medical data repository 120 (e.g., EMRs). In another example, the workflow server 125 may receive an indication from the physician device 105 or the collection entity 115 about a pathology case which may have associated digital slides stored in the medical data repository 120. With the pathology case already identified, any associated digital slides may be requested and received.

It is noted that throughout the functionalities of the workflow server 125 and any information being used by the workflow server 125, manual inputs or information provided by a manual entry may be incorporated. For example, a physician of a patient may provide a manual input as to a clinical question or a reason for which a sample is taken. Specifically, this manual input may be entered with an EMR of the patient. The information that is manually entered may also indicate the type of sample, when the sample was taken, how the sample was taken (e.g., instruments or technique used), how the sample was preserved, a priority level, a deadline, etc. In another example, instead of relying on an automated determination by the workflow server 125, a manual input may be entered that may supersede any automated determination. In this manner, the corresponding pathology case may include additional information from manual inputs that may be used by the workflow server 125.

In utilizing the manual inputs, the workflow server 125 may be configured with various functionalities to interpret the inputs. In a first example, the manual inputs may be provided using standardized forms or in another manner of input substantially similar to a form input. In this way, the workflow server 125 may receive the form and identify a selection being made corresponding to a particular piece of information. In a second example, the workflow server 125 may be configured with natural language processing or parsing operations to receive free-form text that may be analyzed to determine the information contained in the manual input. In a third example, the workflow server 125 may be configured to normalize a manual input (e.g., into keywords) to identify the information contained in the manual input.

Upon receiving the digital slides (and any accompanying information associated with the digital slides or identified pathology case), the analysis engine 240 may be configured to perform its functionality. As noted above, the analysis engine 240 may receive and analyze the digital slides to generate additional information. Specifically, the digital slides may be analyzed to determine case characteristics (e.g., organ/tissue type, extraction method, time that the sample is ready for dispatch to a pathologist for diagnosis, a number of slides, a priority level, a deadline, etc.) and/or predicted case characteristics (e.g., expected diagnosis time, required additional tests, difficult of case assessment, etc.). The analysis engine 240 may utilize any available information (e.g., based on information stored in the medical data repository 120 or based on information determined by the workflow server 125) in generating this additional information.

The analysis engine 240 may be further configured with a functionality to use the available information in determining other characteristics used by the workflow server 125. In a first example, the analysis engine 240 may determine pathologist characteristics. Specifically, using historical pathology case information that may be stored in the medical data repository 120, the analysis engine 240 may identify characteristics associated with the available pathologists that may be assigned to the pathology case. The pathologist characteristics may include specialties, availability, role, etc. In a second example, the analysis engine 240 may determine implicit or explicit information on a status of tasks in a workflow. As will be described in further detail below, a workflow may be selected for a pathology task where the workflow may include at least one task. Each of these tasks may be tracked and a status of the task may be determined (e.g., task has started, task has completed, task is on hold, etc.).

Using the available information, the additional information, and any other source of information, a workflow to be used for a pathology case may be determined. As noted above, the selection engine 250 may determine a workflow to be associated with a pathology case. Also noted above, the selection engine 250 may utilize the workflow repository 130 which stores a plurality of workflows that may be used in completing a pathology case. It is noted that the pathology case may utilize one or more workflows. For illustrative purposes, the exemplary embodiments are described with respect to using a single workflow. However, another iteration of the process associated with a single workflow may be used for any further workflow that may be associated with the pathology case.

In a particular implementation of the selection engine 250 selecting a workflow, the generated case characteristics derived using the analysis engine 240 (e.g., based on the digital slides) may enable selection and execution of a workflow. For example, the digital slides (and an indicated clinical question) may be used to detect that a likely diagnosis of a HER2 IHC slide is equivocal. Based on this determination, a workflow may be selected that pre-orders a HER2 FISH test to increase an expected diagnosis time of a pathology case. This improved efficiency may extend to an assigned pathologist such as if the pathologist may wish to read the IHC slide as well as the results of the FISH test. In another example, the digital slides may be used to detect a likely difficulty of the pathology case (e.g., based on an expected diagnosis outcome). Depending on a policy of the pathology entity (which is described in detail below), the selection engine 250 may trigger a non-routine workflow such as a teaching workflow, a workflow where a dedicated expert (e.g., non-local) is involved, a quality assurance workflow where more than one pathologist is assigned for the same task (to review concordance), etc. It is noted that quality assurance workflows may be used for subsequent pathology case analyses, especially if the diagnosis of the pathology case type is likely to be difficult.

Those skilled in the art will understand that there are many mechanisms that may be used in determining a workflow to be used. With regard to the exemplary embodiments, based on the information provided by the analysis engine 240, the selection engine 250 may perform the determination of the workflow. For example, the analysis engine 240 may analyze the digital slides and the information associated with the pathology case to determine one or more keywords or determine the clinical question to be resolved by the pathology case. The workflows stored in the workflow repository 130 may be associated with keywords or linked to one or more clinical questions. In this manner, the selection engine 250 may determine the workflow that is to be associated with the digital slides.

It is note that the workflow repository 130 may be populated with workflows that are created and/or updated in a variety of different manners. For example, the workflow server 125 may be configured to generate one or more workflows based on available information such as completed, historical pathology cases. In another example, the workflow repository 130 may be populated with workflows created by an administrator. In a further example, the workflow server 125 may be configured to utilize current available information in updating the workflows stored in the workflow repository 130 (e.g., to reflect a particular pathology entity, a region, etc.).

The selection engine 250 may further determine tasks that are associated with the selected workflow. According to the exemplary embodiments, upon identifying the workflow that is associated with the pathology case to which the digital slides correspond, the selection engine 250 may determine the tasks that are to be completed for this workflow. For example, the selection engine 250 may utilize the available information as well as any determined information (e.g., output from the analysis engine 240) to determine the manner in which the workflow is to be performed via the tasks necessary to use the workflow given the particular conditions/criteria of the pathology case. In a particular example, the identified workflow may be used for a particular manner of diagnosing a sample. Based on this workflow and the clinical question (that may be provided or determined), the selection engine 250 may determine the tasks that are needed. In this manner, the tasks associated with the workflow may be identified.

It is noted that the workflow may be associated with a different sets of tasks. That is, a given workflow may not necessarily use the same set of tasks from one pathology case to another pathology case. For example, a first pathology case and a second pathology case may include digital slides of a common type of organ. Accordingly, the same workflow may be selected. However, the first pathology case may relate to a first clinical question while the second pathology case may relate to a second, different clinical question. Therefore, despite using the same workflow, the tasks that are to be performed for the workflow may be different. However, it is also noted that a workflow may include a base set of tasks that are always performed when the workflow is selected. In this implementation, each time the workflow is selected, this base set of tasks may always be performed.

It is further noted that the workflow repository 130 may store the tasks associated with the workflows, particularly as the workflow server 125 is used for pathology cases. In a first example, when a workflow includes a base set of tasks, these tasks may be associated with the workflow. In a second example, when a workflow has been selected, the workflow may have a database associated therewith such that conditions/criteria of the pathology case for which the workflow is selected may be stored. In this manner, when the pathology case has been completed, the tasks that were determined to be used for the pathology case may also be stored in the database. Thus, when another pathology case having substantially similar conditions/criteria as a pathology case in the database and when the same workflow is selected, the tasks that were previously selected may again be selected (or at least recommended for selection).

Once the tasks have been determined by the selection engine 250, each task may have a pathologist assigned thereto. In determining which pathologist to select for each task, the workflow server 125 may utilize a plurality of the engines. Specifically, the workflow server 125 may use the assignment engine 235, the planning engine 245, the deployment engine 255, and the efficiency engine 260. As noted above, the planning engine 245 may generate task allocations based on task allocation requests by following assignment rules (e.g., optimization goals, resource availability, etc.). The assignment engine 235 may receive various inputs to select a pathologist to be assigned a task. The deployment engine 255 may determine policies and optimization goals that are to be considered in assigning the pathologist to the task. The efficiency engine 260 may utilize the policies and optimization goals to identify a pathologist to be assigned to the task.

It is noted that when a plurality of tasks is determined for the workflow, there may be one or more pathologists that are assigned for these tasks. For example, a determination may be made by the workflow server 125 that a single pathologist may be assigned to perform the tasks, two different pathologists may be assigned to each perform respective ones of the tasks, or another plurality of different pathologists may be assigned to each perform respective ones of the tasks where each pathologist may perform one or more of the tasks. Accordingly, each of the tasks may be assigned to a different pathologist, all of the tasks may be assigned to a single pathologist, or any assignment determination in between these extremes may be used.

In assigning tasks to pathologists, the deployment engine 255 may perform various operations to define how pathologists are selected in view of optimization goals of the pathology entity. Specifically, the deployment engine 255 may be configured to split rules and goals that define the relevant policies at the pathology entity into atomic models. The atomic models may represent sub-sets of rules that each correspond to a single atomic goal and may be expressed with non-conflicting rules (i.e., to not conflict with another rule). The deployment engine 255 may structure and manage policies of the pathology entity by defining these atomic models that may be combined in complex or composite models as needed.

The atomic model according to the exemplary embodiments may include non-conflicting and non-overlapping rules. The atomic model may also include a corresponding domain model where the domain model may relate to a conceptual model of topics related to a specific problem describing entities, attributes, relationships, etc. in addition to constraints that govern the specific problem. The atomic model may also be linked to a well-defined goal to be optimized. Once defined, the atomic models may be combined into composite models implementing policies for the pathology entity. Comprehensive annotations may be maintained for the atomic models that help detect conflicts and overlaps among the atomic models when attempting to a combination in the composite model as well as identify variations in the domain models to which the atomic models apply. As those skilled in the art will understand, composite models may include conflicting scoring rules with defined scores/weights for each. The exemplary embodiments may utilize the scores to determine a trade-off based on the atomic models to achieve an optimization goal.

As will be described in further detail below, the atomic models and composite models may be developed by the deployment engine 255 based on initial models. Accordingly, the model and rules repository 135 that may store the atomic and composite models may be linked to corresponding optimization goals. In this manner, the atomic and composite models may be efficiently reused and extended to apply to the context of further pathology entities.

Although the deployment engine 260 may operate in a substantially autonomous manner, the deployment engine 260 may also be configured with a user interface enabling a user (e.g., an administrator) to select atomic and/or composite models. In using this feature, the deployment engine 260 may also visualize conflicts among rules (e.g., automatic detection and manual identification) as well as conflicts stored as annotations to be shown when a user attempts to combine the certain atomic or composite models. The deployment engine 260 may further enable rules and scores used with the atomic models in creating the composite models to be inspected and modified if desired. The deployment engine 260 may additionally be configured for new atomic models to be created (manually or automatically). The deployment engine 260 may include an evaluation/validation feature to aid a user in testing the effects of changes in scores/penalties and rules on the desired optimization goals through selections of atomic models and creating composite models.

The deployment engine 260 may perform a plurality of operations to generate policies for a pathology entity. For example, the deployment engine 260 may receive an indication of optimization goals to be achieved by the pathology entity when completing pathology cases. The optimization goals may be processed (e.g., natural language processing or form identification) to determine characterizations or keywords associated with the optimization goals. For example, the optimization goals may be related to efficiency, throughput, fairness, a combination thereof, resource allocation, timeliness, etc.

Based on the optimization goals, the deployment engine 260 may determine the atomic models that correspond to the optimization goals. As described above, the atomic models may be linked to a corresponding goal. For example, a first atomic model may be linked to an efficiency goal. A second atomic model may also be linked to an efficiency goal. A third atomic model may be linked to a fairness goal. Accordingly, the atomic models may be determined and collected for further processing.

As noted above, the atomic models may be combined into a composite model directed to the identified optimization goal. Although the atomic models are developed with non-conflicting and non-overlapping rules, when combined into the composite model, a rule from a first atomic model may conflict with a rule from a second atomic model. Thus, the deployment engine 260 may resolve conflicts that arise from creating the composite model. For example, a scoring operation may be used where a first atomic model associated with a first conflicting rule is analyzed against a second atomic model associated with a second conflicting rule. Specifically, a trade-off may be identified where a composite model that includes or excludes the conflicting atomic model is determined. Subsequently, the better composite model that enables the optimization goal to be achieved is identified. In this manner, the composite model may provide a representation of how the optimization goal may be achieved by incorporating proper atomic models and composite models. The composite model may be used to generate the corresponding policy.

As noted above, the deployment engine 260 may also be used to enable a user interface for manual inputs. Thus, along the process at any stage, a user may provide an input that may supersede any automated determination by the deployment engine 260. For example, the optimization goals may be provided by the user. The optimization goals may be provided in any format (e.g., free-form text, in a standardized form, etc.). In another example, an atomic model or composite model may be selected from a predetermined list of models. When a manual selection is entered, the deployment engine 260 may be configured to incorporate the selected model to the best of its ability relative to the optimization goals. However, if the selected model is ultimately determined to include conflicts that are unresolved or if the optimization goals cannot be met, the deployment engine 260 may return an alert to the user.

The deployment engine 260 may further determine whether optimization goals are capable or incapable of being achieved based on the available atomic and composite models. For example, particularly during an earlier stage of use of the deployment engine 260, there may not be a sufficient list of available atomic or composite models. Accordingly, to achieve a particular optimization goal, a new atomic model may be created that may be included in a composite model for a policy that achieves the optimization goal.

Although the deployment engine 260 may perform the above operations in a substantially automated manner, when the policies are created based on user approval, the deployment engine 260 may generate the policies based on the atomic and composite models as recommendations to be shown to the user. Thus, via the user interface, the user may browse, edit, combine models, etc. based on the recommendations provided by the deployment engine 260.

The deployment engine 260 may additionally be configured with a feedback operation in which results of policies are verified. That is, the deployment engine 260 may determine if a policy that is used based on atomic and composite models has ultimately achieved the corresponding optimization goal. For example, a first policy may have successfully achieved its optimization goal for a first percentage of the time the first policy is selected. In another example, a second policy may have failed to achieve the optimization goal for a second percentage of the time the second policy is selected. Based on these successes and failures, the deployment engine 260 may more accurately determine how atomic and composite models are to be selected for certain policies directed toward an optimization goal. The feedback operation may also be used to annotate conflicts in the models through manual or automated detection. The deployment engine 260 may benchmark effects of changes to policies for subsequent uses in achieving optimization goals.

As noted above, the deployment engine 260 may perform its functionality to develop the atomic and composite models to be used in creating policies to achieve optimization goals. The use of the deployment engine 260 may focus on analyzing the policies and building the atomic models linked to specific optimization goals. Initial uses of the deployment engine 260 in its infancy may build the model and rules repository 135 along with corresponding annotations that may be reused by later uses. As those skilled in the art will understand, the effort to model the policies is larger in earlier uses of the deployment engine 260. Once a strong library of atomic models has been built, the deployment engine 260 may use the repository 135 for subsequent implementations making the work of a modeler more efficient and faster (e.g., composing entire policies out of available building blocks, addressing conflicts by modifying constraints and their trade-offs, only define new models when the available models do not support all set goals and policies of the pathology entity, etc.).

Based on the above implementation of the deployment engine 260, the workflow server 125 may provide a variety of features. For example, the workflow server 125 may be configured to enable an efficient modeling and task implementation for assignment policies in various workflows at the pathology entity. In another example, the workflow server 125 may be configured to provides an intuitive manner to build, evaluate, and test/validate policies in achieving optimization goals. In a further example, the workflow server 125 may be configured to support the process of translating textual policies into computerized models in achieving optimization goals.

The workflow server 125 may also utilize the efficiency engine 260 in assigning tasks to pathologists. Again, the efficiency engine 260 may utilize the policies and optimization goals (e.g., as defined with the deployment engine 255) to identify a pathologist to be assigned to the task. The efficiency engine 260 may perform various operations to select one of the available pathologists to be assigned to a given task to achieve a desired optimization goal. The efficiency engine 260 may also utilize an independent approach or a holistic approach such that a selection of a pathologist for a task may be based on the present conditions or may be based on how a selection affects a schedule of task assignments.

Using digital pathology, diagnosis statistics may be determined. Accordingly, the workflow server 125 may efficiently access and analyze an actual diagnosis time for each pathologist based on case type and identify changes in performance and specialty of the pathologists. In contrast to conventional approaches to completing pathology cases, the analysis of logs of historically completed pathology cases may enable the workflow server 125 according to the exemplary embodiments to move away from using average overall performance attributes and instead use variables that change over time which are dynamically determined. These variables may be assigned values based on a live analysis of the historical logs. While the averages may be used to estimate a resource utilization when fairness (with respect to workload) needs to be preserved among the pathologists, more accurate diagnosis time values that are derived from collected activity data may improve achieving the optimization goals (e.g., throughput, turnaround, etc.). Therefore, the workflow server 125 according to the exemplary embodiments combine the two approaches maintaining fairness (e.g., not rewarding the less efficient pathologists with less work) while finding a schedule that has the potential to yield a desired overall performance.

As will be described in detail below, using collected traces from a workflow (e.g., a portion of the workflow that is completed by a pathologist), the efficiency engine 260 may be configured to evolve timing characteristics describing an expected performance of each pathologist based on the case type and take into account changes of these values over time. The timing characteristics may be used in two types of settings. The statistics may be used to assign pathologists to given pathology cases. For example, the assignment may be for a given pathology entity including a plurality of pathologists and the current pathology cases at the pathology entity that require diagnosis within a period of time. The statistics may also be used to track a performance per pathologist over time.

In providing the above noted functionalities, the efficiency engine 260 may be configured to perform a plurality of operations. In a first operation, the efficiency engine 260 may include a solver functionality. The solver functionality may utilize domain models and scoring rules as well as activity estimate tables and workflow models that describe the manner of operation of a particular pathology entity with key activities selected to use as input parameters in achieving an optimization goal. Accordingly, the solver functionality may estimate a diagnosis time per case type per pathologist. It is noted that the solver functionality may also be extended to cover other activities. It is also noted that the domain model may include parameters and/or variables that may be filled by the activity estimate tables.

In a second operation, the efficiency engine 260 may include a log processing functionality. The log processing functionality may retrieve a log of activities from an image management system (e.g., in the medical data repository 120) that are mapped onto selected activity classes. In a third operation, the efficiency engine 260 may include an analysis functionality. The analysis functionality may process the logs and calculate current values of the activity parameters per pathologist and case type. The analysis functionality may feed the output for further processing (e.g., to update a rules/constraints definition). It is noted that the analysis functionality may include a feedback functionality that processes the outcomes of the changes in estimates and any impact on an optimization goal to be achieved. In a fourth operation, the efficiency engine 260 may include an optimization functionality. The optimization functionality may take into account the domain model and the rules definition to propose a solution for the pathology case assignment that optimizes the desired optimization goal (e.g., throughput or turnaround).

To determine a selection of a pathologist to be assigned a task of the workflow for a pathology case, the efficiency engine 260 may receive and review historical logs of previous pathology cases and associated tasks. Specifically, workflow traces may be collected for pathologists reading pathology cases/tasks along with corresponding case features. The features (e.g., the clinical question, the type of organ, number and type of pathology slides associated to a pathology case, the disease, etc.) may be used to construct the case types. In this manner, the pathologist, the case type, and the corresponding task may be linked to determine an estimate of a likely time that the pathologist may require in performing a similar task for a similar case type. Specifically, the above operations may be used to calculate the expected reading time for each case type per pathologist. Additionally, the above operations may be used to calculate timing estimations for other relevant activities that may be used as predictors.

As noted above, there may be a window of time in which a schedule of tasks require diagnosis by a pathologist. Based on these known, scheduled tasks, the efficiency engine 260 may utilize the expected times required by each of the pathologists for each task. Then, based on any optimization goal that is to be met and any policies that may be defined (e.g., via the deployment engine 255), the efficiency engine 260 may select a pathologist for each of the tasks. In this manner, the efficiency engine 260 may use these estimations to compute schedules for the tasks where the one considered to achieve any defined optimization goals or policies based on a set of key performance indicators may be executed. For each pathology case, the efficiency engine 260 may give a mean value for estimated effort (e.g., in time units) per each eligible pathologist that is used to decide to which pathologist the pathology case/task is to be assigned.

It is noted that the above functionality is based on historical pathology cases and a current expected time that the pathologists will need in diagnosis a particular case type or task. However, for an initial iteration when a sufficient source of data is unavailable, the efficiency engine 260 may use deployment values that are assigned to an expected effort per pathology case. The deployment values may be based on averages, any available data, or a combination thereof. Thus, while the system is running and the efficiency engine 260 is being used, the deployment values or any updated values may be updated (or further updated) as traces (e.g., tasks completed in historical pathology cases) are collected.

As noted above, the efficiency engine 260 may include a feedback functionality. Accordingly, when updating the values, actual timing/effort for each pathology case and task may be compared with the determined estimated values used in assigning the task to a pathologist. The estimated (e.g., predicted) values for the assigned pathologist and case type may thereby be updated. A standard deviation may also be computed and used to determine whether a change to the estimated diagnosis time is required (e.g., outliers may be detected and ignored). The parameters/variables may be updated in the domain model based on an analysis of the historical logs according to defined policies (e.g., update average diagnosis duration for case type after every 200 new cases of that type and exclude outliers). It is noted that the updating operation may be performed based on any timing criteria. For example, a predetermined interval based on time or number of pathology cases/tasks may be used. In another example, a dynamic interval may be used. In a further example, a constant updating may be used as each pathology case/task is completed.

Based on the above implementation of the efficiency engine 260, the workflow server 125 may provide a variety of features. For example, the workflow server 125 may be configured to assignment determinations indicating the pathologist who is to be assigned a particular task of a pathology case. The assignment determination may be optimized by including knowledge about the expected performance of each pathologist given the case type. This is used for accurate prediction of turnaround and throughput in the optimization functionality. In another example, the workflow server 125 may be configured to track the performance of a pathologist, thereby allowing for a comparison with peers and comparing the performance of a pathologist in different periods. In a further example, the workflow server 125 may be configured to detect and take into account changes in performance of pathologists for subsequent iterations.

Once the tasks have been assigned to a pathologist, the workflow server 125 may dispatch the assignments. The exemplary embodiments may utilize any dispatch mechanism for each pathologist to perform the respective duties in completing the task of the workflow for the pathology case. For example, if the pathology case is performed by a pathology entity (e.g., in a pathology department of a hospital, in a lab, etc.), the workflow server 125 may provide the assignments to the pathology entity. The pathology entity may schedule the assignments (e.g., based on any scheduling requirements in the assignments).

Once each of the tasks of the workflow have been completed, the exemplary embodiments may also be configured with a feedback operation in which the results or subsequent actions are also received by the workflow server 125. Based on the feedback data, the workflow server 125 may update any information in the medical data repository 120, may update the workflows/tasks stored in the workflow repository 130, and/or may update the models/rules stored in the model and rules repository 135. In a particular manner of using the feedback operation, pathologist characteristics may be updated in which the most current experience is incorporated to reflect a profile of the pathologist. Thus, any further pathology case that is processed may rely on a contemporary profile of each pathologist.

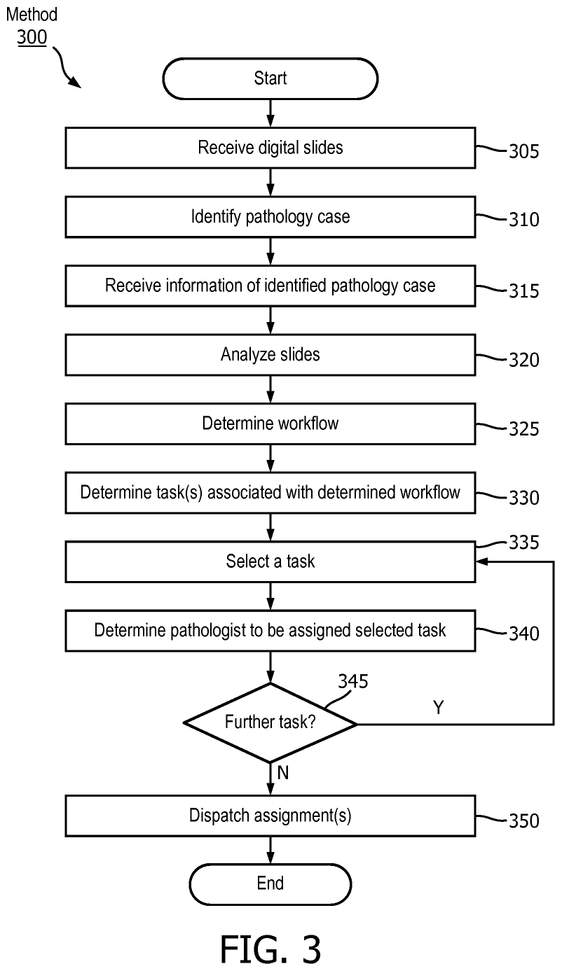

FIG. 3 shows an overall method 300 for automatically completing a pathology case according to the exemplary embodiments. Specifically, the method 300 relates to determining the manner in which a workflow for a pathology case is divided into tasks that are assigned to pathologists. The method 300 also relates to how the workflow server 125 generates and/or utilizes additional information that is gained from using digital pathology slides in a digital pathology procedure. The method 300 will be described from the perspective of the workflow server 125 and the engines 235-260. The method 300 will also be described with regard to the system 100 of FIG. 1 and the workflow server 125 of FIG. 2.

In 305, the workflow server 125 receives digital slides associated with a pathology case. As described above, the digital slides may be digital files of samples of a patient captured by a collection entity 115. For example, the samples may be a tissue or body fluid. The collection entity 115 (or another organization) may digitize the sample into digital slides that may be stored in the medical data repository 120. The workflow server 125 may be provided the digital slides in a direct manner from the collection entity 115 (or organization that created the digital slides) or may request the digital slides from the medical data repository 120. It is noted that the workflow server 125 may have received an indication of the pathology case to which the digital slides are associated in a variety of manners (as described above). Accordingly, the workflow server 125 may have been prompted to receive the digital slides based on an identified pathology case.

In 310, the workflow server 125 identifies the pathology case to which the digital slides are associated. If the identification of the pathology case was used in retrieving the digital slides, the workflow server 125 may have already identified the pathology case. However, if the workflow server 125 had received the digital slides without the identification, the workflow server 125 may analyze the digital slides (e.g., via the analysis engine 240) to determine various characteristics associated with the digital slides. For example, the type of sample may be identified. Based on the characteristics, the workflow server 125 may reference the information stored in the medical data repository 120 (e.g., EMRs) to identify the pathology case. In 315, based on the identified pathology case, the workflow server 125 may receive associated information. For example, the clinical question associated with the pathology case may be included in the associated information.

In 320, the workflow server 125 via the analysis engine 240 may analyze the digital slides. As noted above, the workflow server 125 is configured to determine additional information from the digital slides to be used in determining how to use a workflow in completing the pathology case. Specifically, the digital slides may be analyzed to determine case characteristics (e.g., organ/tissue type, extraction method, time that the sample is ready for dispatch to a pathologist for diagnosis, a number of slides, etc.) and/or predicted case characteristics (e.g., expected diagnosis time, required additional tests, difficult of case assessment, etc.).

In 325, based on the received information associated with the digital slides and the additional information derived from the digital slides, the workflow server 125 determines a workflow to be used in completing the pathology case. For example, the workflow server 125 may access the workflow repository 130 that stores a plurality of workflows that may be used to complete a pathology case. In 330, the workflow server 125 determines the one or more tasks associated with the determined workflow. As noted above, the tasks may be intimately linked with the workflow. That is, select tasks may always be used when the corresponding workflow is selected. There may also be other tasks that are dynamically selected for the corresponding workflow, for example, based on the received and/or additional information of the digital slides.

In 335-340, the workflow server 125 via the planning engine 245 and the selection engine 250 select a task and determine a pathologist to be assigned the selected task. The selection of the pathologist will be described in further detail with regard to the deployment engine 255 and the efficiency engine 260. In 345, the workflow server 125 determines if there are any further tasks that require a pathologist to be assigned. If another task is present in the workflow, the workflow server 125 returns to 335. However, when all tasks have been assigned a pathologist, in 350, the workflow server 125 dispatches the assignments such as in a schedule for an upcoming window of tasks to be completed by a pathology entity.

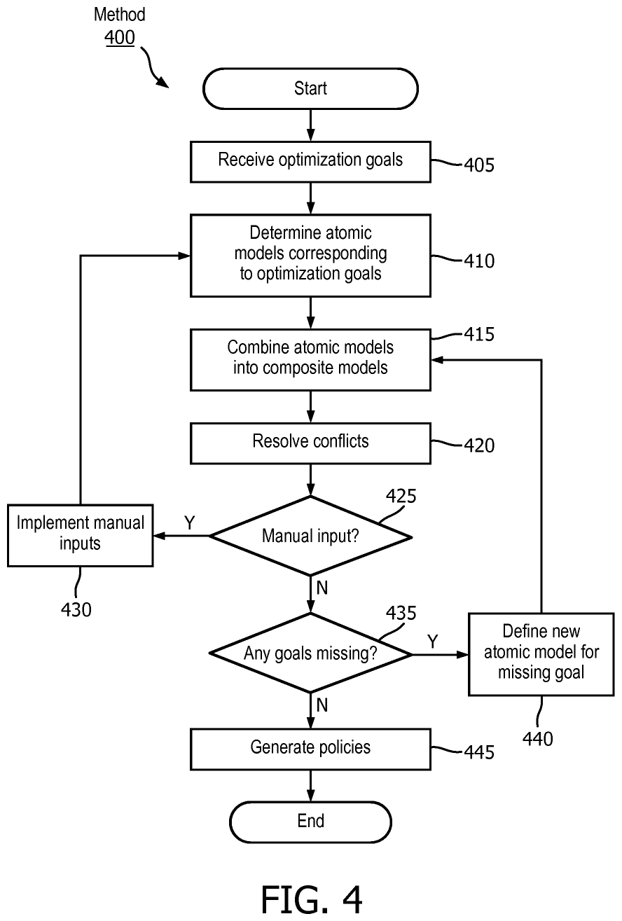

FIG. 4 shows a method 400 for generating policies for assigning a pathologist to a pathology case according to the exemplary embodiments. Specifically, the method 400 may relate to generating and utilizing atomic models in creating composite models that form the basis of the policies to achieve optimization goals at a pathology entity. The method 400 will be described from the perspective of the workflow server 125 and the deployment engine 255. The method 400 will also be described with regard to the system 100 of FIG. 1 and the workflow server 125 of FIG. 2.

In 405, the workflow server 125 receives optimization goals for the pathology entity. For example, the optimization goals may relate to throughput, turnaround, fairness, etc. The optimization goals may be received manually from a user such as an administrator of the pathology entity or may be determined automatically based on predetermined criteria at which the pathology entity is configured to operate. Based on the optimization goals, in 410, the workflow server 125 determines the atomic models corresponding to the optimization goals where the atomic models are defined with non-conflicting and non-overlapping rules. As described above, the workflow server 125 may access the model and rules repository 135 that stores atomic models, each of the atomic models being associated with one or more optimization goals. Thus, the atomic models corresponding to the optimization goals may be determined. It is noted that the method 400 is described herein where the model and rules repository 135 have been populated with atomic models from at least one prior use of the deployment engine 255.

In 415, the workflow server 125 combines the atomic models corresponding to the optimization goals into one or more composite models. As noted above, although the atomic models may include rules that may stand independently and in combination, the composite models through combination of atomic models may result in conflicts among rules included in the atomic models. Thus, in 420, the workflow server 125 resolves the conflicts. For example, a tradeoff may be determined between atomic models to improve the manner in which the optimization goal may be achieved.

In 425, the workflow server 125 may determine if a manual input has been received. Initially, it is noted that the manual input may be received at any stage along the method 400. For example, the manual input may be the optimization goals. In another example, the manual input may be atomic models that are manually selected. A manual input may take precedence over automatically determined inputs. Thus, if a manual input is received, in 430, the workflow server 125 implements the manual input and returns to 410 where the atomic models are determined and in 415, composite models are created.

If no manual input is received, in 435, the workflow server 125 determines if the composite models having any conflict resolved results in any of the optimization goals not being achieved. Particularly when the model and rules repository 135 has not been fully built to include the appropriate models, a combination into a composite model may not be capable of achieving the optimization goals that are received. If at least one optimization goal is not achievable with the composite model, in 440, the workflow server 125 defines a new atomic model for the missing goal that may be incorporated into a composite model. Thereafter, the workflow server 125 returns to 415 where the composite model including the new atomic model is assessed to resolve conflicts in 420. When all the optimization goals are achievable with the composite models including atomic models, in 445, the policies corresponding to the optimization goals are generated.

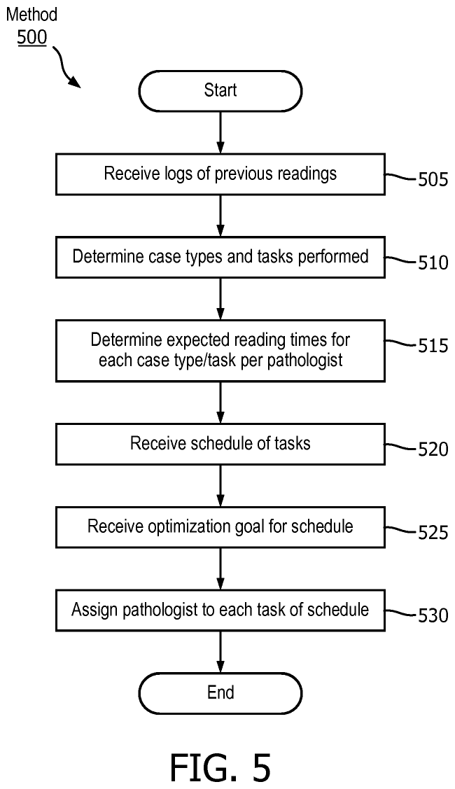

FIG. 5 shows a method 500 for assigning a pathologist to a pathology case according to the exemplary embodiments. Specifically, the method 500 may relate to utilizing policies and optimization goals along with historical pathology case/task information associated with pathologists. The method 500 will be described from the perspective of the workflow server 125 and the efficiency engine 260. The method 500 will also be described with regard to the system 100 of FIG. 1 and the workflow server 125 of FIG. 2.

In 505, the workflow server 125 receives logs of previous pathology cases/tasks that have been completed. The logs may include various types of information. Thus, in 510, the workflow server 125 determines the case types and tasks that were performed in the logs. The case types may be based on various characteristics (e.g., organ type). The logs may also include the corresponding pathologist who was assigned the completed task as well as timing information regarding an amount of time that the pathologist used in completing the task. Accordingly, in 515, the workflow server 125 may determine an expected reading time to complete a specific case type or task by a particular pathologist. In maintaining the expected times of the pathologist/case type, the workflow server 125 may determine schedules in completing upcoming tasks.

In 520, the workflow server 125 receives a schedule of upcoming tasks. As noted above, the workflow server 125 may determine the tasks to be assigned to pathologists for a window of time. For example, the workflow server 125 may determine the tasks to be performed on a given day at a future time (e.g., a week from a current day). The tasks may be associated with one or more workflows of one or more pathology cases. It is noted that the use of the window of time for tasks is only exemplary. In another exemplary embodiment, the workflow server 125 may determine the tasks to be assigned to pathologists based on a workflow for a specific pathology case. Thus, the tasks associated with the pathology case may have pathologists assigned thereto.

In 525, the workflow server 125 receives the optimization goals for the schedule of tasks. As noted above, the optimization goals may have policies associated therewith and include atomic and/or composite models in achieving the optimization goals. Thus, based on the estimates from performing 505-515 and also based on the optimization goals/policies, in 530, the workflow server 125 assigns a pathologist to each task for the schedule to achieve the optimization goals with a greatest probability of success.

The exemplary embodiments provide a device, system, and method of completing a pathology case by optimizing the manner in which a workflow is to be completed. By utilizing additional information gleaned from digital slides of a digital pathology procedure, tasks that are to be completed in the workflow of the pathology case may be determined. In this manner, the exemplary embodiments may assign one or more pathologists to the tasks in an efficient manner. When viewing from a window of time in which tasks are to be completed, the exemplary embodiments may also assign one or more pathologists to the tasks in the window in an efficient manner. Thus, optimization goals of a pathology entity of the pathologists may be achieved.

Those skilled in the art will understand that the above-described exemplary embodiments may be implemented in any suitable software or hardware configuration or combination thereof. An exemplary hardware platform for implementing the exemplary embodiments may include, for example, an Intel x86 based platform with compatible operating system, a Windows platform, a Mac platform and MAC OS, a mobile device having an operating system such as iOS, Android, etc. In a further example, the exemplary embodiments of the above described method may be embodied as a computer program product containing lines of code stored on a computer readable storage medium that may be executed on a processor or microprocessor. The storage medium may be, for example, a local or remote data repository compatible or formatted for use with the above noted operating systems using any storage operation.

It will be apparent to those skilled in the art that various modifications may be made in the present disclosure, without departing from the spirit or the scope of the disclosure. Thus, it is intended that the present disclosure cover modifications and variations of this disclosure provided they come within the scope of the appended claims and their equivalent.

1. A method, comprising:
at a workflow server:
receiving a plurality of digital slides associated with a pathology case, the pathology case associated with first information characterizing the pathology case;
generating second information based on an analysis of the digital slides, the second information characterizing the digital slides;
determining a plurality of tasks used in completing the pathology case based on the first and second information;
generating policies for assigning a pathologist to a task;
determining a task performer to be assigned to perform a select one of the tasks based on the generated policies; and
dispatching an assignment to the task performer corresponding to the selected task.

2. The method of claim 1, wherein the second information includes one of case characteristics, predicted case characteristics, or a combination thereof.

3. The method of claim 2, wherein the case characteristics comprises one of an organ type, a tissue type, an extraction method, a time that a sample of one of the digital slides is ready for dispatch to the task performer, a number of the digital slides, a priority level, a deadline, or a combination thereof.

4. The method of claim 2, wherein the predicted case characteristics comprises one of an expected diagnosis time, required additional tests, a difficulty of case assessment, or a combination thereof.

5. The method of claim 1, wherein the first information includes a clinical question associated with a reason for a sample being drawn to generate the digital slides.

6. The method of claim 1, further comprising:
determining a workflow used in completing the pathology case, the workflow associated with the tasks.

7. The method of claim 1, further comprising:
receiving an optimization goal used in completing the pathology case, the determining the tasks and the task performer being further based on the optimization goal.

8. The method of claim 7, wherein the optimization goal is associated with one of throughput, turnaround, fairness, resource utilization, timeliness, or a combination thereof.

9. The method of claim 7, wherein the determining the task performer is based on the optimization goal being defined in a policy.

10. The method of claim 9, further comprising:
determining a plurality of atomic models corresponding to the optimization goal, the atomic models including non-conflicting and non-overlapping rules.

11. The method of claim 10, further comprising:
combining the atomic models into a composite model representing the policy.

12. The method of claim 11, wherein a first one of the atomic models in the composite model includes first rules and a second one of the atomic models in the composite model includes second rules, a first one of the first rules conflicting with a second one of the second rules.

13. The method of claim 12, further comprising:
determining a scoring value for the first atomic model and the second atomic model in achieving the optimization goal using the composite model;
determining a respective tradeoff of including the first atomic model, the second atomic model, or both the first and second atomic models in the composite model; and
identifying the respective tradeoff having a highest probability of achieving the optimization goal.

14. The method of claim 1, wherein the determining the task performer is based on logs of historical completed pathology cases, each historical completed pathology case including a respective plurality of completed tasks, each completed task associated with a respective task performer.

15. The method of claim 14, further comprising:
determining an expected reading time of the task performer for the selected task, the expected reading time based on the logs.

16. A workflow server, comprising:
a transceiver communicating via a communications network, the transceiver configured to receive a plurality of digital slides associated with a pathology case, the pathology case associated with first information characterizing the pathology case;
a memory storing an executable program; and
a processor that executes the executable program that causes the processor to perform operations, comprising:
generating second information based on an analysis of the digital slides, the second information characterizing the digital slides;
determining a plurality of tasks used in completing the pathology case based on the first and second information;
generating policies for assigning a pathologist to a task;
determining a task performer to be assigned to perform a select one of the tasks based on the generated policies; and
dispatching an assignment to the task performer corresponding to the selected task.

17. The workflow server of claim 16, wherein the second information includes one of case characteristics, predicted case characteristics, or a combination thereof.

18. The workflow server of claim 17, wherein the case characteristics comprises one of an organ type, a tissue type, an extraction method, a time that a sample of one of the digital slides is ready for dispatch to the task performer, a number of the digital slides, a priority level, a deadline, or a combination thereof.

19. The workflow server of claim 17, wherein the predicted case characteristics comprises one of an expected diagnosis time, required additional tests, a difficulty of case assessment, or a combination thereof.

20. A method, comprising:
at a workflow server:
receiving a plurality of digital slides associated with a plurality of pathology cases, the pathology cases associated with respective first information characterizing the corresponding pathology case;
generating second information based on an analysis of the digital slides, the second information characterizing the digital slides;
determining a plurality of tasks to be completed in a window of time, the plurality of tasks associated with the pathology cases based on the first and second information;
generating policies for assigning a pathologist to a task;
determining a task performer to be assigned to perform a select one of the tasks based on the generated policies; and
dispatching an assignment to the task performer corresponding to the selected task,
wherein the assignment is associated with an optimization goal of one of fairness, throughput, turnaround, resource allocation, timeliness, or a combination thereof.
